(19) United States
(12) Patent Application Publication
(10) Pub. No.: US 2012/0263791 A1
(43) Pub. Date: Oct. 18, 2012

(54) FOMULATION COMPRISING 1 H-QUINAZOLINE-2, 4-DIONE AMPA RECEPTOR ANTAGONISTS, IN THE FORM OF IMMEDIATE RELEASE TABLETS AND PREPARATION THEREOF

(75) Inventors: Joseph L. Zielinski, Florham Park, NJ (US); John Vrettos, Union, NJ (US); Qin Ji, Randolph, NJ (US); Subash Patel, Somerville, NJ (US)

(73) Assignee: NOVARTIS AG, BASEL (CH)

(21) Appl. No.: 13/518,027

(22) PCT Filed: Dec. 21, 2010
(86) PCT No.: PCT/US10/61553

§ 371 Date: Jun. 21, 2012

Related U.S. Application Data
(60) Provisional application No. 61/288,985, filed on Dec. 22, 2009.

(51) Int. Cl. A61K 9/28; A61P 25/00; A61K 31/517

(52) U.S. Cl. 424/474; 514/266.3; 514/266.23

(57) ABSTRACT

The present invention relates to pharmaceutical formulations comprising a 1H-quinazoline-2,4-dione AMPA receptor antagonist. More particularly, the invention relates to immediate release formulations comprising such a compound.

### FIELD OF THE INVENTION

The present invention relates to pharmaceutical formulations comprising a 1H-quinazoline-2,4-dione AMPA receptor antagonist. More particularly, the invention relates to immediate release formulations comprising such a compound.

### BACKGROUND TO THE INVENTION

WO 2006/108591 and equivalent U.S. application Ser. No. 11/911,040, both included herein in their entirety (including in particular the examples) by reference for all purposes, describe 1H-quinazoline-2,4-diones and their use in the treatment of any pathology, disorder or clinical condition involving altered AMPA receptor function or AMPA receptor mediated neuronal damage. More particularly, there are described compounds of the formula (I):

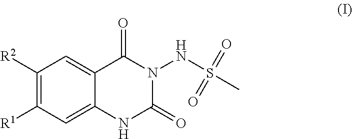

wherein
R¹ represents CF₃, CHF₂, CH₂F, CH₃CHF—, CH₃CF₂—, ethyl or iso-propyl and
R² represents alkyl substituted by one or more substituents, the substituents being selected from the group consisting of halogen, nitro, cyano, acyl, hydroxy, oxo (═O), alkoxy, cycloalkoxy, acyloxy, alkoxycarbonyloxy, amino, alkylamino, dialkylamino, formyl, acylamino, alkoxycarbonylamino or
R² represents heterocyclylalkyl substituted by one or more substituents, the substituents being selected from the group consisting of halogen, nitro, cyano, hydroxy, alkoxy, alkylcarbonyloxy, alkoxycarbonyloxy, amino, alkylamino, dialkylamino, alkoxycarbonylamino, or
R² represents phenyl substituted by one or more substituents, the substituents being selected from the group consisting of cyano, hydroxy, alkanediyl, alkenediyl, alkoxy, hydroxyalkyl, formyl, alkylcarbonyl, alkoxycarbonyl, alkylcarbonyloxy, alkoxycarbonyloxy, amino, alkylamino, dialkylamino, aminoalkyl, alkylaminoalkyl, dialkylaminoalkyl, alkoxycarbonylamino, or
R² represents heterocyclyl optionally substituted by one or more substituents, the substituents being selected from the group consisting of halogen, hydroxy, amino, nitro, cyano, alkyl, hydroxalkyl, alkoxyalkyl, aminoalkyl, alkylaminoalkyl, dialkylaminoalkyl, acyl, alkoxy, acyloxy, alkoxycarbonyloxy, amino, alkylamino, dialkylamino, acylamino, alkoxycarbonylamino and whereby the heterocycle is bound to the phenyl ring by a carbon-atom
   and their salts.

A formulation comprising one or more members of the above class of drugs would be desirable with immediate release providing rapid absorption after taking the one or members of the class of drugs. Immediate release formulations are known in pharmaceutical technology. However, one formulation cannot be used for all drugs since the formulations have to be individually designed for each active ingredient or class of active ingredients. The behaviour of a specific medicinal substance (class) when combined with one or more immediate release excipients cannot be calculated or generally predicted. Interactions between the immediate release material(s) on the one hand and the active ingredient on the other can affect drug release as well as the processing and storage properties of the formulation. A particular difficulty which arises with the aforesaid drug class arises in devising a composition capable of forming a cohesive tablet which does not fall apart too easily. Another particular difficulty which arises with the aforesaid drug class arises in devising a composition capable of forming a tablet which shows satisfactory values for friability and/or hardness range. Another particular difficulty which arises with the aforesaid drug class arises in devising a composition which achieves a desirably high level of drug loading in a tablet.

It would therefore be particularly desirable to provide a tablet capable of having a relatively high drug loading and of being cohesive.

### SUMMARY OF THE INVENTION

According to the invention there is provided a tablet comprising a compound of Formula (I) or a salt thereof and a hydroxypropylcellulose.

Another aspect of the invention is a tablet comprising a compound of Formula (I) or a salt thereof, a hydroxypropylcellulose and a disintegrant.

The active ingredient (compound or salt) may be in an amount of from 2.5% to 30% by weight of the tablet, calculated excluding any coating, for example from 5% to 27.5%, such as e.g. 5% to 26%. In one embodiment, the active ingredient is in an amount of from 20% to 26%.

The hydroxypropylcellulose may be in an amount of from 2% to 10% by weight of the tablet, calculated excluding any coating, for example from 3% to 8%, such as e.g. 4% to 7% or 3% to 5%.

In embodiments, the tablets further contain a water-insoluble filler and a water soluble filler.

In one aspect, the invention provides a process for making a tablet by wet granulation, characterised in that the internal phase ingredients comprise a hydroxypropylcellulose and a compound of Formula (I) or a salt thereof.

The invention includes uncompressed compositions for making the tablets of the invention and comprising a compound of Formula (I) or a salt thereof and a hydroxypropylcellulose. The disclosure includes also a homogeneous composition comprising a compound of Formula (I) or a salt thereof and a hydroxypropylcellulose in powder or tablet form. The invention provides also a powder blend comprising a compound of Formula (I) or a salt thereof and a hydroxypropylcellulose, which blend may be compressed to form tablets.

Further aspects and embodiments of the invention are set forth in the following description and claims.

### Description of Various Embodiments

The following definitions are used herein:

HPC=hydroxypropylcellulose

HPMC=hydroxypropylmethylcellulose

MCC=microcrystalline cellulose

Tablet core=the body of a tablet excluding any film coating, i.e. the product resulting from tablet compression.

The following description discloses various numerical values qualified by the word “about”. The description also includes the same numerical values when not qualified by the word “about”.

In one aspect, the invention provides a tablet comprising a compound of Formula (I) or a salt thereof. The formulation is characterised by comprising a hydroxypropylcellulose.

The invention includes a tablet comprising a therapeutically effective amount of a compound of Formula (I) or a salt thereof and a hydroxypropylcellulose and having immediate release of the active ingredient.

The term “immediate release” refers in particular to release of the active ingredient within about two hours of contact with actual or simulated gastric fluid, in particular within about one hour of contact with actual or simulated gastric fluid. In particular immediate release tablets may commence release of the active ingredient within about 30 minutes of contact with actual or simulated gastric fluid.

The term “therapeutically effective amount” refers to an amount which achieves a therapeutic effect.

In one embodiment, the active ingredient is a compound of Formula (I) and not a salt thereof. In another embodiment, the active ingredient is a salt of a compound of Formula (I). In one embodiment, the compound of Formula (I) or salt thereof is a hydrate or other solvate. In another embodiment, the compound of Formula (I) or salt thereof is not a hydrate or other solvate. Lists of suitable pharmaceutically acceptable salts are found in Remington's Pharmaceutical Sciences, 17th ed., Mack Publishing Company, Easton, Pa., US, 1985, p. 1418, the disclosure of which is hereby incorporated by reference; see also Stahl et al, Eds, “Handbook of Pharmaceutical Salts Properties Selection and Use”, Verlag Helvetica Chimica Acta and Wiley-VCH, 2002.

A particular HPC which may be used in the invention is a low molecular weight HPC, e.g. with a nominal molecular weight of less than about 150 kDa, for example of less than about 100 kDa, and in particular of about 80 kDa. The HPC may have a molecular weight of at least about 70 kDa. In an embodiment, the HPC is EXF HPC, namely a low molecular weight HPC with a fine particle size and a nominal molecular weight of 80 kDa.

The tablets may contain, in addition to the hydroxypropylcellulose (“HPC”) and the active ingredient, a water soluble filler. As water soluble filler may be mentioned one or a combination of potassium carbonate, sodium carbonate, ammonium carbonate, calcium lactate, mannitol, urea, inositol, magnesium succinate, sorbitol, and carbohydrates such as, for example, mannitol, raffinose, sucrose, glucose, fructose, lactose and lactose monohydrate. A particular water soluble filler is lactose monohydrate.

In embodiments, the tablets contain a compactable filler, e.g. MCC.

In embodiments, the tablets contain a water insoluble filler, e.g. MCC.

The disclosure includes embodiments in which the tablets contain a disintegrant, for example croscarmelose sodium or sodium starch glycolate.

The tablets may further comprise one or more other excipients, for example one or more of a lubricant, a glidant and a flow aid. As examples of lubricants may be mentioned stearic acid, calcium stearate, magnesium stearate or talc, or a combination thereof. As examples of glidants may be mentioned dried aluminium hydroxide gel or magnesium silicate, or a combination thereof. As an exemplary flow aids there may be mentioned silica, e.g. anhydrous colloidal silica.

The invention includes an embodiment in which the tablet is film-coated.

An exemplary, tablet, therefore, comprises:
an active compound (compound, not salt)
an HPC
a water soluble filler, e.g. lactose monohydrate
a water insoluble filler, e.g. microcrystalline cellulose.

Such an exemplary tablet may further comprise other tabletting excipient(s), e.g. a flow aid, (for example silica), a disintegrant (for example sodium starch glycolate) and a tablet lubricant (e.g. magnesium stearate). The tablet may further include an optional film coating.

A particular tablet of the invention, therefore, comprises or consists of the following: active compound, 80 kDa HPC, lactose monohydrate, microcrystalline cellulose, silica, sodium starch glycolate and magnesium stearate (or in variants another tablet lubricant), as well as an optional film coating.

A single tablet suitably contains from about 1 mg to about 500 mg of active principle, e.g. from about 5 mg to about 250 mg of active principle In some embodiments, a single tablet contains from about 10 mg to about 200 mg of active ingredient. A particular content of active principle is from about 20 mg to about 200 mg active principle per tablet. Some tablets contain from about 50 mg to 150 mg active ingredient per tablet.

There will next be described the proportions of the different ingredients in embodiments of the invention. Needless to say, the total percentage contents of the ingredients of the formulations add up to 100%.

In embodiments, the active ingredient constitutes from about 2.5% to about 60% of the tablet core (i.e. the tablet excluding any optional coating) by weight, such as from about 2.5% to about 30% of the tablet core (i.e. the tablet excluding any optional coating) by weight e.g. from about 5% to about 27.5% of the tablet by weight. A particular embodiment contains from about 20 weight % to about 30 weight %, e.g. about 20 weight % active ingredient to about 26 weight % to active ingredient, all the aforesaid percentages being calculated on the basis of the weight of the tablet core. A further particular embodiment contains from about 24 weight % to about 28 weight % of active ingredient, e.g. about 25 weight % to about 27 weight % of active ingredient, e.g. about 26% weight % of active ingredient, all the aforesaid percentages being calculated on the basis of the weight of the tablet core. A further particular embodiment contains from about 25 weight % to about 55 weight % of active ingredient, all the aforesaid percentages being calculated on the basis of the weight of the tablet core.

The HPC is suitably in amount of from about 2.9% to about 10% by weight of the tablet core, e.g. about 3% to 8% by weight of the tablet core, such as about 3% to 5% by weight of the tablet core.

The disclosure includes tablets which comprise a water insoluble filler, e.g. MCC, in an amount of from about 15% to about 60%, such as 20% to about 60%, for example about 25% to about 50%. In some tablets, the water insoluble filler is in an amount of from 25% to 45%, e.g. 35% to 45%, as for example in the case of tablets containing about 35.4%, about 43.8% or about 36.3% water insoluble filler. The percentages in this paragraph are percentages by weight of the tablet core, i.e. by weight of the tablet excluding any coating film.

Tablets of the invention may contain a disintegrant in an amount of from about 1% to about 20% by weight calculated on the basis of the weight of the tablet core, e.g. about 2.5% to about 15%, as in the case of tablets containing about 4% to about 12% disintegrant e.g.

about 4% to about 6% disintegrant. In one embodiment, the disintegrant is in an amount of from about 5% to about 10% by weight of the tablet core, e.g. about 5% to about 7%, as in the case of tablets containing 5% or 6.8% disintegrant by weight calculated on the basis of the weight of the tablet core.

Other excipients, e.g. a flow aid or lubricant, may be present in amount of up to about 10% by weight calculated on the basis of the weight of the tablet core, e.g. about 1% to about 10%. In one embodiment, other excipients are present in an amount of from about 1.5% to about 5%, e.g. about 2% to about 4%, as in the case of tablets containing about 2.1%, 2.4% or 3.7% of other excipients.

Further embodiments are described in the following Table 1:

TABLE 1

| | Embodi- | Embodi- | Embodi- |
|---|---|---|---|
| | ment 1 | ment 2 | ment 3 |
| | Weight % | Weight % | Weight % |
| Ingredient | of Tablet | of Tablet | of Tablet |
| Active compound | 2.5%-30% | 5%-27.5% | 5%-26% |
| HPC (e.g. 80 kDa | 2%-10% | 3%-8% | 4%-7% |
| HPC) | | | |
| Soluble filler | 20%-40% | 25%-35% | 27%-32% |
| (e.g. lactose | | | |
| monohydrate) | | | |
| Insoluble filler | 25%-50% | 25%-45% | 35%-45% |
| (e.g. microcrys- | | | |
| talline cellulose) | | | |
| Disintegrant | 2.5%-15% | 4%-12% | 5%-10% |
| Other excipient(s) | Up to 10%, | Up to 5%, | Up to 4% |
| (e.g. flow acid, | e.g. 1-10% | e.g. 1.5%-5% | e.g. 2%-4% |
| lubricant etc) | | | |

The above weight percentages exclude the optional coating.

In a particular sub-embodiment, the active compound is in an amount of from about 20% to about 26% by weight of the tablet core, the HPC is in an amount of from about 3% to about 5% of the tablet core, the water soluble filler in an amount of from about 25% to about 30% by weight of the tablet core, the disintegrant is an amount of from about 4% to about 6% by weight of the tablet core, and other excipients are in an amount of from about 3% to about 4% of the tablet core.

For all embodiments of the invention, the following combination of features forms one optional sub-class: the HPC has a molecular weight of from about 70 kDa to about 100 kDa, e.g. about 80 kDa; the water insoluble filler is microcrystalline cellulose; the water soluble filler is lactose monohydrate; and the disintegrant is sodium starch glycolate.

The other excipients typically include, or are, a flow aid (e.g. silica) and a tablet lubricant (e.g. magnesium stearate).

The total weight of a tablet core is suitably of from 100 mg to 1g, e.g. 175 mg to 900 mg, e.g. is from 180 mg to 220 mg, from 350 mg to 450 mg or from 750 mg to 850 mg.

Active Compound

The active ingredient is a compound of Formula (I) previously mentioned or a salt thereof. For example, the compound of Formula (I) may be described in WO 2006/108591 in any of Examples 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 4 0, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80 and 81. Structures of some exemplary active ingredients are below:

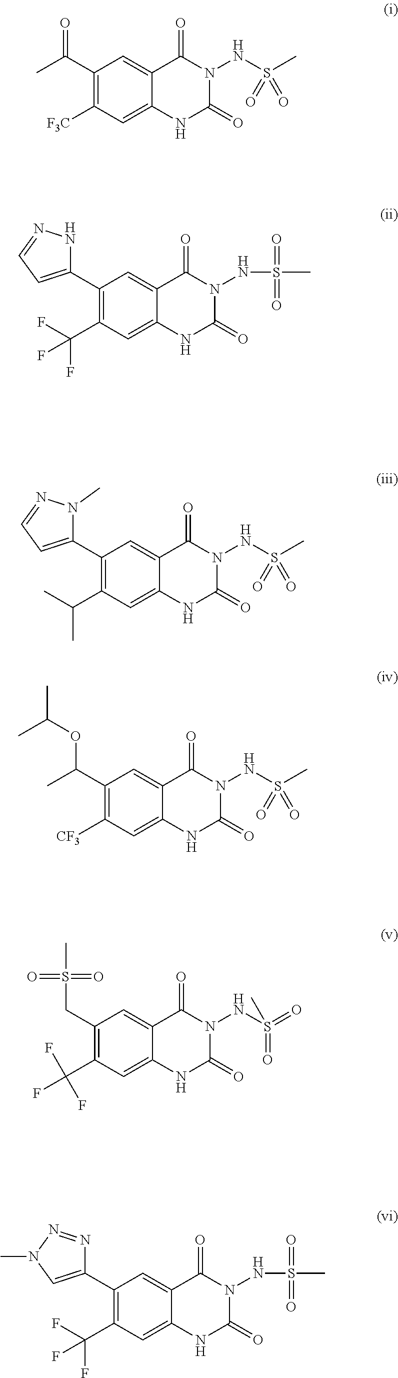

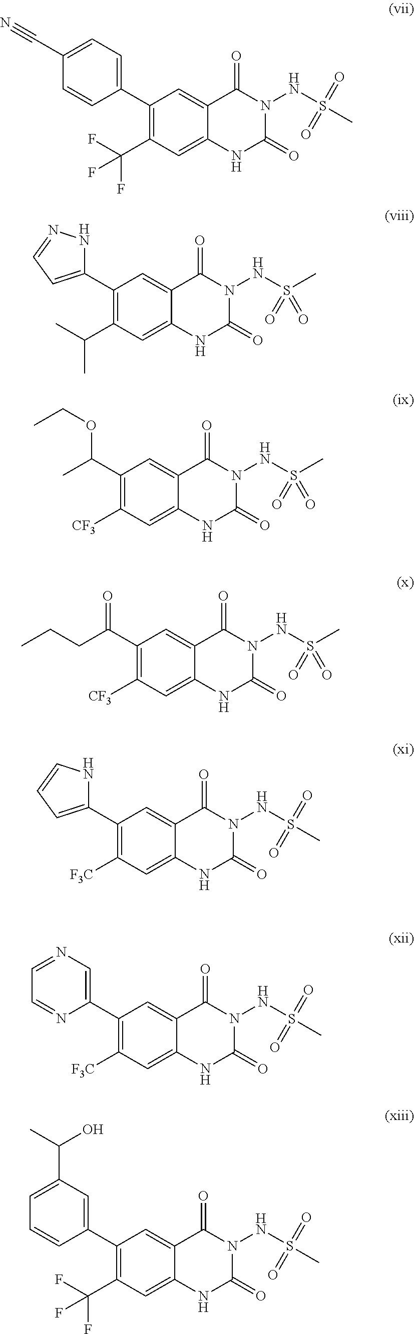

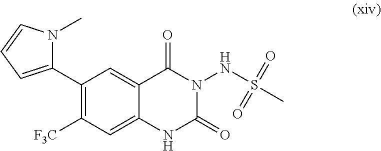

The active ingredient is useful for treating epilepsy, psychosis in schizophrenia, in bipolar disorder, in Parkinson's disease and in drug-induced psychosis and in postictal psychosis, neurodegenerative disorders (such as multiple sclerosis, amyotrophic lateral sclerosis, Parkinson's Disease, Huntington's Disease or Alzheimer's Disease), schizophrenia, esp. chronic schizophrenia, anxiety, depression, bipolar mood disorders, sleep disorders, cognitive disorders, emesis, tinnitus, pain, neuronal pain, migraine, anesthetics, myopia, tumor growth, withdrawal symptoms, ischemic and hypoxic conditions (such as stroke, subarachnoid haemorrhage, perinatal hypoxia, brain and spinal cord trauma, head injury, high intracranial pressure, and any surgical procedure potentially associated with hypoxia of the central nervous system), and conditions produced by the actions of environmental, exogenous neurotoxins, including those produced by infections as well as those produced by metabolic changes and hepatic encephalopathy associated with liver failure. Formulations of the invention are therefore useful for treating such disorders, e.g. epilepsy.

Method of Preparation

The tablets of the invention may be made by wet granulation. More specifically, the constituents of the formulation may be mixed with one another at the same time or in a specific sequence and granulated by moistening with water and drying the resultant granular mass. If the mixture is granulated, it is optionally milled and, whether or not milled, is suitably screened to a desired particle size. Extragranular excipients, e.g. fillers, flow agents and lubricants can be added to the granules after granulation. More particularly, the inner phase components comprising the active ingredient, the matrix former(s) and the optional water soluble filler are mixed and wet granulated. After drying, the granulate is mixed with the outer phase components to form a dry mixture.

The dry mixture may be compressed to form tablets. The compressed tablets may be coated with one or more films, if desired.

The invention therefore provides in one embodiment a process for preparing tablets, comprising:
preparing a wet granulate of an internal phase by combining water and the solid constituents of an internal phase, said solid constituents comprising a hydroxypropylcellulose and a compound of Formula (I), and granulating the combination of the water and the solid constituents;
drying the granulate;
blending the dry granulate with the constituents of an external phase to form a tabletting mixture; and
compressing the tabletting mixture.

The pre-compression mixture is included in the invention, which therefore includes a homogeneous composition comprising a compound of Formula (I) or a salt thereof and a hydroxypropylcellulose in powder form. Alternatively stated, the invention includes a powder blend comprising a compound of Formula (I) or a salt thereof and a hydroxypropylcellulose.

In more detail, an exemplary procedure for manufacturing the tablets comprises following steps:

Step 1: Place the components of the internal phase, i.e. the drug substance, the binder(s), and the filler(s) and any other internal phase constituents, into a mixer, e.g. into the bowl of the high shear mixer.

Step 2: Mix (e.g., 5 minutes).

Step 3: Add water to the mixture of step (2).

Step 4: Mix/knead/granulate the resultant composition.

Step 5 (optional): Screen the wet granulate (e.g., a sieve of 2 mm mesh size).

Step 6: Dry the granulate, e.g. on trays or preferably in a fluid bed dryer (preferred).

Step 7: Screen external phase, i.e. the filler(s), disintegrant(s), glidant(s)/flow agent(s), lubricant(s) and dried granulate into a blender (e.g., a sieve of 1 mm mesh size).

Step 8: Blend the components of step (7).

Step 9: Compress the tabletting mixture of step (8), e.g. on a force feeding (rotary) tabletting machine to tablet cores of the required weight and dimensions.

Step 10: (optional) Film-coat the tablet, e.g. with an HPMC-based film.

The amount of water used in the wet granulation step (e.g. in step (3) of above steps (1)-(10)) is suitably of from about 25% to about 40% by weight of the solid constituents (i.e. the mixed solids resulting from step (2) in the case of above steps (1)-(10)), e.g. from about 30% to about 35%, from example about 30%, about 31%, about 32%, about 33%, about 34% or about 35%.

The water may suitably be added to the internal phase components over a period of from about 1 minute to about 60 minutes, e.g. about 2 to about 10 minutes, for example over a period of about 5 minutes.

In the present invention, optional step 5 is preferably excluded. In contrast, step 10 is usually included. The invention therefore includes a method for making a formulation of the invention which comprises performing all of steps 1-10 except step 5. It will be understood that variables such as timing, drying technique, mesh size and the constituents of the film coating can be changed and do not have to be as illustrated above.

The external phase components may suitably be added sequentially, one at a time, and blended with the mixture before the next component is added.

In one embodiment, the dried granulate is screened prior to blending with the external phase components and, in particular, may be milled and then screened prior to blending with the external phase components. In one embodiment, the dried granulate is milled prior to blending with the external phase components and, in particular, may be milled and then screened prior to blending with the external phase components.

In exemplary embodiments, therefore, the internal phase used in preparation of the granulate comprises or consists of:
active compound (compound, not salt)
HPC, e.g. 80 kDa HPC
water soluble filler, e.g. lactose monohydrate
water insoluble filler, e.g. microcrystalline cellulose, and the external (extragranular) phase comprises or consists of:
water insoluble filler (e.g. microcrystalline cellulose)
disintegrant (e.g. sodium starch glycolate)
tablet lubricant (e.g. Mg stearate)
flow aid (e.g. silica).

The above-described procedures (e.g. steps 1-4 and 6-9) may be applied to the manufacture of tablets using the ingredients described in the following Table 2:

TABLE 2

| | | | |
|---|---|---|---|
| | Embodi- | Embodi- | Embodi- |
| | ment A | ment B | ment C |
| | Weight % | Weight % | Weight % |
| Ingredient | of Tablet | of Tablet | of Tablet |

INTERNAL PHASE

| | | | |
|---|---|---|---|
| Active compound | 2.5%-30%, | 5%-27.5%, | 5%-26% |
| | e.g. 2.5%-26% | e.g. 5%-26% | |
| HPC (e.g. 80 kDa | 2%-10% | 3%-8% | 4%-7% |
| HPC) | | | |
| Soluble filler | 20%-40% | 25%-35% | 27%-32% |
| (e.g. lactose | | | |
| monohydrate) | | | |
| Insoluble filler | 15%-40% | 20%-35% | 22%-33% |
| (e.g. microcrys- | | | |
| talline cellulose, | | | |
| particularly | | | |
| Avicel ®101) | | | |
| Disintegrant (e.g. | 1.25%-7.5% | 2%-6% | 2.5%-5% |
| sodium starch | | | |
| glycolate) | | | |

EXTERNAL PHASE

| | | | |
|---|---|---|---|
| Insoluble filler | 5%-25% | 8%-17.5% | 12%-15% |
| (e.g. microcrys- | | | |
| talline cellulose, | | | |
| particularly | | | |
| Avicel ® PH102 | | | |
| Flow aid (e.g. | 0%-5%, | 0.25%-3% | 0.5%-2.5% |
| colloidal silica) | e.g. 0.1%-5% | | |
| Disintegrant (e.g. | 1.25%-7.5% | 2%-6% | 2.5%-5% |
| sodium starch | | | |
| glycolate) | | | |
| Tablet lubricant | 1%-3% | 2%-3% | 1.5%-2% |
| (e.g. Mg stearate | | | |

The percentages set out in Table 2 are calculated without including the optional coating, i.e. are calculated on the basis of the table core.

The film-coating may be—and normally is—applied to the compressed core.

In experiments conducted so far, it has been difficult to achieve drug loading of greater than 26% by weight of the dry ingredients of the tablet core (i.e. the tablet excluding any optional coating).

It will be appreciated from the aforegoing that the invention includes a process for the preparation of a drug loaded compressed tablet containing a compound of Formula (I), e.g. in an amount of up to about 26 weight % based on the materials used in the following step (a), the process comprising the following steps: (a) blending the compound with a hydroxypropylcellulose, a water soluble filler, an water insoluble filler and a disintegrant, (b) adding water in an amount of from about 25% to about 40% by weight of the materials used in step (a) over a period of from 1 to 60 minutes to wet granulate the blended mixture and agglomerate it; (c) drying the agglomerated mixture, (d) milling the dried mixture to a granulate and optionally screening it; (e) blending the milled mixture with, in any order, a water insoluble filler, a flow aid, a disintegrant and a tablet lubricant; and (f) compressing the lubricated mixture to a compressed tablet of the desired shape. The compressed tablet may then be film coated.

In an aspect of the invention, the compositions of the invention exhibit good processability for example through a large hardness range. This means that the tablet can be compressed to form a tablet having acceptable tabletting properties (e.g. tablet breaking force e.g. as evaluated using test USP No. 1217 and/or friability e.g. as evaluated using test USP No. 1217) using a broad range of compression forces. For example, in some aspects, the composition tablet may exhibit a hardness range of greater than 25N, for example greater than 30N, such as greater than 40N or greater than 50N. In a preferred embodiment, the tablet exhibits a hardness range greater than 70N. The hardness range may also be less than 100N, for example less than 90N. In some embodiments, the hardness range is less than 80N, for example less than 70N, less than 60N or 50N.

The tabletted compositions of the invention exhibit beneficial friablity properties. Friability is the ability of the tablet to withstand abrasion in packaging, handling and shipping. Friability may be measured by any suitable measuring technique e.g. according to USP standard No. 1216.

In an aspect of the invention, there is provided an immediate release composition comprising compound (iii), for example in a once daily amount of 150 mg (optionally administrable simultaneously as three 50 mg doses). This composition, when administered to a patient may exhibit a release profile such that the AUC_{INF} is in the range of about 17000±30% hr*ng/mL, for example 17000±20% hr*rig/mL , such as 17000±10% hr*ng/mL.

### EXAMPLES

The following examples are applicable to each compound disclosed in WO 2006/108591, for example the compounds of Formula (I) disclosed in the examples thereof, e.g. a compound of structure (i), (ii), (iii), (iv), (v), (vi), (vii), (viii), (ix), (x), (xi), (xii), (xiii) or (xiv).

### Example 1

The following composition is wet granulated following steps 1-4 and 6-8 above and used as a common blend for 25, 50, 100 and 200 mg dose compressed tablets.

| Materials | Qnty/Tab (mg) | Qnty/batch (g) | % w/w |
|---|---|---|---|
| Active compound | 200 | 500 | 26.0 |
| Lactose monohydrate | 211 | 527.5 | 27.5 |
| Avicel ® 101 | 169.5 | 423.75 | 22.1 |
| HPC EXF | 31 | 77.50 | 4.0 |
| Sodium starch glycolate | 19 | 47.5 | 2.5 |
| Internal phase weight | | 1576.25 | |
| EXTERNAL PHASE | | | |
| Avicel ® PH102 | 102.5 | 256.25 | 13.3 |
| Aerosil ® | 4 | 10.00 | 0.5 |
| Sodium starch glycolate | 19 | 47.50 | 2.5 |
| Mg Stearate | 12 | 30.00 | 1.6 |
| Total Weight: | 768 | 1920.00 | 100.0 |

Avicel® 101 and Avicel® PH102 are each a microcrystalline cellulose. Aerosil® is an anhydrous colloidal silica.

### Example 2

The following composition is wet granulated following steps 1-4 and 6-8 above and compressed into 10 mg dose tablets.

| Materials | Qnty/Tab (mg) | Qnty/batch (g) | % w/w |
|---|---|---|---|
| Active compound | 10 | 25 | 5.2 |
| lactose monohydrate | 61 | 152.5 | 31.8 |
| Avicel ® | 60 | 150.00 | 31.3 |
| HPC EXF | 12 | 30.00 | 6.3 |
| Sodium starch glycolate | 9 | 22.50 | 4.7 |
| Internal phase weight | | 380.00 | |
| EXTERNAL PHASE | | | |
| Avicel ® PH102 | 24 | 60.00 | 12.5 |
| Aerosil ® | 4 | 10.00 | 2.1 |
| sodium starch glycolate | 9 | 22.50 | 4.7 |
| Mg stearate | 3 | 7.50 | 1.6 |
| Total Weight: | 192 | 480.00 | 100.0 |

### Example 3

The following composition is wet granulated following steps 1-4 and 6-8 above and compressed into tablets.

| Materials | Qnty/Tab (mg) | Qnty/batch (g) | % w/w |
|---|---|---|---|
| Active compound | 200 | 500 | 25.0 |
| lactose monohydrate | 219 | 547.5 | 27.4 |
| Avicel ® 101 | 177.5 | 443.75 | 22.2 |
| HPC EXF | 32 | 80.00 | 4.0 |
| sodium starch glycolate | 20 | 50.00 | 2.5 |
| Internal phase weight | | 1621.25 | |
| EXTERNAL PHASE | | | |
| Avicel ® PH102 | 112.5 | 281.25 | 14.1 |
| Aerosil ® | 4 | 10.00 | 0.5 |
| sodium starch glycolate | 20 | 50.00 | 2.5 |
| Mg Stearate | 15 | 37.50 | 1.9 |
| Total Weight | 800 | 2000.00 | 100.0 |

### Examples 4-11

The compositions of Examples 4-10 were prepared using a wet granulation technique. Compound (iii) was blended together with the excipients of the internal phase in a top driven granulator mixer (Gral). Water (at 24 to 30%) was then sprayed into the mixer and the resulting composition granulated over 2 to 10 minutes. The granules were then dried using a fluid bed dryer or tray drier until a moisture level of 2 to 3% was achieved. The excipients of the external phase and the dried granules were screened using a 1mm screen and then mixed in a tumble blender. The lubricant (Mg stearate) was then screened using a 0.5mm screen and added to the granulation and blended in a tumble blender.The blend was then compressed on a force feeding (rotary) tabletting machine to convert the mixture into tablet form. The tablet was then film coated using Opadry II brown.

The composition of Example 11 was prepared using a roller compaction technique as described in this example. The compositions used in these wet granulation and roller compaction methods are specified in Examples 4-11. In these Examples the following abbreviations are used: wet granulation=WG; roller compaction=RC; Compound of formula (iii)=Compound (iii).

### Example 4

Composition TRD-2689-33.

| Materials | Qnty/Tab (mg) | Qnty/Tab (mg) | % w/w |
|---|---|---|---|
| Compound (iii) | 200 | 50 | 26.0 |
| lactose monohydrate | 211 | 77.7475 | 27.5 |
| Avicel ® 101 | 169.5 | 42.375 | 22.1 |
| HPC EXF | 31 | 7.75 | 4.0 |
| sodium starch glycolate | 19 | 4.75 | 2.5 |
| (Explotab) | | | |
| Internal phase weight | 730.5 | 182.6225 | |
| EXTERNAL PHASE | | | |
| Avicel ® PH102 | 102.5 | 25.625 | 13.3 |
| Aerosil ® | 4 | 1 | 0.5 |
| sodium starch glycolate | 19 | 4.75 | 2.5 |
| (Explotab) | | | |
| Mg Stearate | 12 | 3 | 1.6 |
| Total Weight | 768 | 216.9975 | 100.0 |

### Example 5

Composition TRD-2689-13.

| Materials | Qnty/Tab (mg) | Qnty/Tab (mg) | % w/w |
|---|---|---|---|
| Compound (iii) | 200 | 50 | 50.0 |
| lactose monohydrate | 75 | 18.75 | 18.8 |
| HPC EXF | 16 | 4 | 4.0 |
| sodium starch glycolate | 10 | 2.5 | 2.5 |
| (Explotab) | | | |
| Internal phase weight | 301 | 75.25 | |
| EXTERNAL PHASE | | | |
| Avicel ® PH102 | 79 | 19.75 | 19.8 |
| Aerosil ® | 2 | 0.5 | 0.5 |
| sodium starch glycolate | 10 | 2.5 | 2.5 |
| (Explotab) | | | |
| Mg Stearate | 8 | 2 | 2.0 |
| Total Weight | 400 | 100 | 100.0 |

### Example 6

Composition TRD-2689-16.

| Materials | Qnty/Tab (mg) | Qnty/Tab (mg) | % w/w |
|---|---|---|---|
| Compound (iii) | 200 | 50 | 40.0 |
| lactose monohydrate | 125 | 31.25 | 25.0 |
| HPC EXF | 16 | 4 | 3.2 |
| sodium starch glycolate | 12 | 3 | 2.4 |
| (Explotab) | | | |
| Internal phase weight | 353 | 88.25 | |
| EXTERNAL PHASE | | | |
| Avicel ® PH102 | 121.5 | 30.375 | 24.3 |
| Aerosil ® | 2.5 | 0.625 | 0.5 |
| sodium starch glycolate | 13 | 3.25 | 2.6 |
| (Explotab) | | | |
| Mg Stearate | 10 | 2.5 | 2.0 |
| Total Weight | 500 | 125 | 100.0 |

### Example 7

Composition TRD-2689-17.

| Materials | Qnty/Tab (mg) | Qnty/Tab (mg) | % w/w |
|---|---|---|---|
| Compound (iii) | 200 | 50 | 30.3 |
| lactose monohydrate | 171 | 42.75 | 25.9 |
| Avicel ® 101 | 131 | 32.75 | 19.8 |
| HPC EXF | 33 | 8.25 | 5.0 |
| sodium starch glycolate | 16 | 4 | 2.4 |
| (Explotab) | | | |
| Internal phase weight | 551 | 137.75 | |
| EXTERNAL PHASE | | | |
| Avicel ® PH102 | 76 | 19 | 11.5 |
| Aerosil ® | 3 | 0.75 | 0.5 |
| sodium starch glycolate | 17 | 4.25 | 2.6 |
| (Explotab) | | | |
| Mg Stearate | 13 | 3.25 | 2.0 |
| Total Weight | 660 | 165 | 100.0 |

### Example 8

Composition TRD-2689-19.

| Materials | Qnty/Tab (mg) | Qnty/Tab (mg) | % w/w |
|---|---|---|---|
| Compound (iii) | 200 | 50 | 50.0 |
| lactose monohydrate | 71 | 17.75 | 17.8 |
| PVP K30* | 20 | 5 | 5.0 |
| sodium starch glycolate | 10 | 2.5 | 2.5 |
| (Explotab) | | | |
| Internal phase weight | 301 | 75.25 | |
| EXTERNAL PHASE | | | |
| Avicel ® PH102 | 79 | 19.75 | 19.8 |
| Aerosil ® | 2 | 0.5 | 0.5 |
| sodium starch glycolate | 10 | 2.5 | 2.5 |
| (Explotab) | | | |
| Mg Stearate | 8 | 2 | 2.0 |
| Total Weight | 400 | 100 | 100.0 |

### Example 9

Composition TRD-2677-145.

| Materials | Qnty/Tab (mg) | Qnty/Tab (mg) | % w/w |
|---|---|---|---|
| Compound (iii) | 200 | 50 | 50.0 |
| lactose monohydrate | 66 | 16.5 | 16.50 |
| Avicel ® PH102 | 66 | 16.5 | 16.50 |
| HPMC 2910 3 cps | 28 | 7 | 7.00 |
| croscarmellose Na | 15 | 3.75 | 3.75 |
| CMC XL (Acid-sol) | | | |
| Internal phase weight | 375 | 93.75 | |
| EXTERNAL PHASE | | | |
| Aerosil ® 200 | 2 | 0.5 | 0.50 |
| croscarmellose Na | 15 | 3.75 | 3.75 |
| CMC XL (Acid-sol) | | | |
| Mg Stearate | 8 | 2 | 2.00 |
| Total Weight | 400 | 100 | 100.0 |

### Example 10

Composition TRD-2577-130.

| Materials | Qnty/Tab (mg) | Qnty/Tab (mg) | % w/w |
|---|---|---|---|
| Compound (iii) | 200 | 50 | 50.0 |
| mannitol DC Partek M200 | 148 | 37 | 20.56 |
| Avicel ® PH102 | 166 | 41.5 | 23.06 |
| HPMC 2910 3 cps | 51 | 12.75 | 7.08 |
| croscarmellose Na | 29 | 7.25 | 4.03 |
| CMC XL (Acid-sol) | | | |
| Internal phase weight | 594 | 148.5 | |
| EXTERNAL PHASE | | | |
| Avicel ® PH102 | 80 | 20 | 11.11 |
| Aerosil ® | 2 | 0.5 | 0.28 |
| croscarmellose Na | 29 | 7.25 | 4.03 |
| CMC XL (Acid-sol) | | | |
| Ca Stearate | 15 | 3.75 | 2.08 |
| Total Weight | 400 | 100 | 100.0 |

### Example 11

Composition TRD-2577-135.

| Materials | Qnty/Tab (mg) | Qnty/Tab (mg) | % w/w |
|---|---|---|---|
| Compound (iii) | 200 | 50 | 18.52 |
| mannitol DC Partek M200 | 310 | 77.5 | 28.70 |
| Avicel ® PH102 | 360 | 90 | 33.33 |
| PVP K30* | 110 | 27.5 | 10.19 |
| croscarmellose Na | 40 | 10 | 3.70 |
| CMC XL (Acid-sol) | | | |
| Internal phase weight | 1020 | 255 | |
| EXTERNAL PHASE | | | |
| croscarmellose Na | 40 | 10 | 3.70 |
| CMC XL (Acid-sol) | | | |
| Ca Stearate | 20 | 5 | 1.85 |
| Total Weight | 1080 | 270 | 100.0 |

This formulation was roller compacted (RC) and compressed into tablet form using the procedure below:

The internal phase excipients (Compound (iii), mannitol, Avicel 102, PVP K30, and croscarmellose) were screened using a 1mm screen and then blended together using a tumble blender. This powder blend then was roller compacted. The resultant material was then screened using a 1mm screen. The external phase excipients, croscarmellose was screened and blended together. The lubricant, calcium stearate, was then screened using a 0.5 mm screen and blended to produce the final granulation for tablet compression.

### Example 12

The physical properties of the tablets of Examples 4 -11 were tested and the results given below.

| | Tabletting | | Lower tabletting | Lower tabletting | |
|---|---|---|---|---|---|
| | (WG = wet | | force, upper | force, upper | |
| | granulation; | | tabletting force and | tabletting force and | |
| | RC = roller | | hardness range (N) | hardness range (N) | Drug |
| | compaction) | friability | for 200 mg tablet | for 50 mg tablet | load % |
| TRD-2689-33 | WG | pass | 150 N, 230 N, 80 N | 78 N, 106 N, 28 N | 26% ca. |
| TRD-2689-13 | WG | pass | 95 N, 128 N, 33 N | NA | 50% |
| TRD-2689-15 | WG | pass | 160 N, 200 N, 40 N | 50 N, 75 N, 25 N | 40% |
| TRD-2689-17 | WG | NA | NA | NA | 30% |
| TRD-2689-19 | WG | NA | NA | NA | 50% |
| TRD-2577-145 | WG | NA | NA | NA | 50% |
| TRD-2577-130 | WG | fail | 45 N, 90 N, 45 N | 20 N, 30 N, 10 N | 27.78% |
| TRD-2577-135 | RC | fail | NA | NA | 19.6% |

Hardness range (a measure of the processability of the tablet composition) was assessed using a Dr Schleuniger Tablet tester, model 8M. The minimum force necessary to form a tablet having acceptable tabletting properties (e.g. tablet breaking force e.g. as measured using the USP No 1217 breaking test) is given as “Lower tabletting force”. The maximal force, still forming a tablet having these properties is given as the “upper tabletting force”. The difference is given as the “hardness range”.

Friability was tested using USP No. 1216. The tablet weight was measured before and after the test and a visual inspection for breakages made. The tablet sample passed the friability test if it exhibited <0.8% loss in weight and without breakage of tablets.

For compositions TRD-2689-33, TRD-2689-13, TRD-2689-15 the force values are the values required to produce a tablet satisfying the breaking force test (USP No. 1217) and the friability test (USP No 1217). For composition TRD-2577-130, the force values are the values required to produce a tablet satisfying the breaking force test (USP No. 1217) but not the friability test (USP No 1217).

### Example 13

A 50 mg tablet as specified in Example 4 was prepared using the procedure described in Example 4. Three tablets were administered in a single dose to a sample population (12) and the PK parameters measured. These parameters are provided below.

Primary Analysis

Primary PK variables were long-transformed and analyzed using a mixed effects linear model. Sequence, Period and Treatment factors were included in the model as fixed effects and subject as a random effect. 90% confidence intervals for PK variables (pharmacokinetic analysis population).

Drug Substance: Compound (iii); Matrix: Plasma; Analyte: Compound (iii).

| PK variable (unit) | Adjusted geometric mean |
|---|---|
| AUCinf [hr*ng/mL] | 16813.83 |
| AUClast [hr*ng/mL] | 16807.09 |
| Cmax [ng/mL] | 2908.266 |

1. A tablet comprising a hydroxypropylcellulose and an active ingredient selected from compounds of Formula (I) and salts thereof: 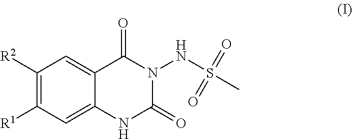
wherein
R¹ represents CF₃, CHF₂, CH₂F, CH₃CHF-, CH₃CF₂—, ethyl or iso-propyl and
R² represents alkyl substituted by one or more substituents, the substituents being selected from the group consisting of halogen, nitro, cyano, acyl, hydroxy, oxo (═O), alkoxy, cycloalkoxy, acyloxy, alkoxycarbonyloxy, amino, alkylamino, dialkylamino, formyl, acylamino, alkoxycarbonylamino or
R² represents heterocyclylalkyl substituted by one or more substituents, the substituents being selected from the group consisting of halogen, nitro, cyano, hydroxy, alkoxy, alkylcarbonyloxy, alkoxycarbonyloxy, amino, alkylamino, dialkylamino, alkoxycarbonylamino, or
R² represents phenyl substituted by one or more substituents, the substituents being selected from the group consisting of cyano, hydroxy, alkanediyl, alkenediyl, alkoxy, hydroxyalkyl, formyl, alkylcarbonyl, alkoxycarbonyl, alkylcarbonyloxy, alkoxycarbonyloxy, amino, alkylamino, dialkylamino, aminoalkyl, alkylaminoalkyl, dialkylaminoalkyl, alkoxycarbonylamino, or
R² represents heterocyclyl optionally substituted by one or more substituents, the substituents being selected from the group consisting of halogen, hydroxy, amino, nitro, cyano, alkyl, hydroxalkyl, alkoxyalkyl, aminoalkyl, alkylaminoalkyl, dialkylaminoalkyl, acyl, alkoxy, acyloxy, alkoxycarbonyloxy, amino, alkylamino, dialkylamino, acylamino, alkoxycarbonylamino and whereby the heterocycle is bound to the phenyl ring by a carbon-atom.

2. . A tablet of claim 1, wherein the compound is selected from one of the compounds of the formula (i) to (xiv):

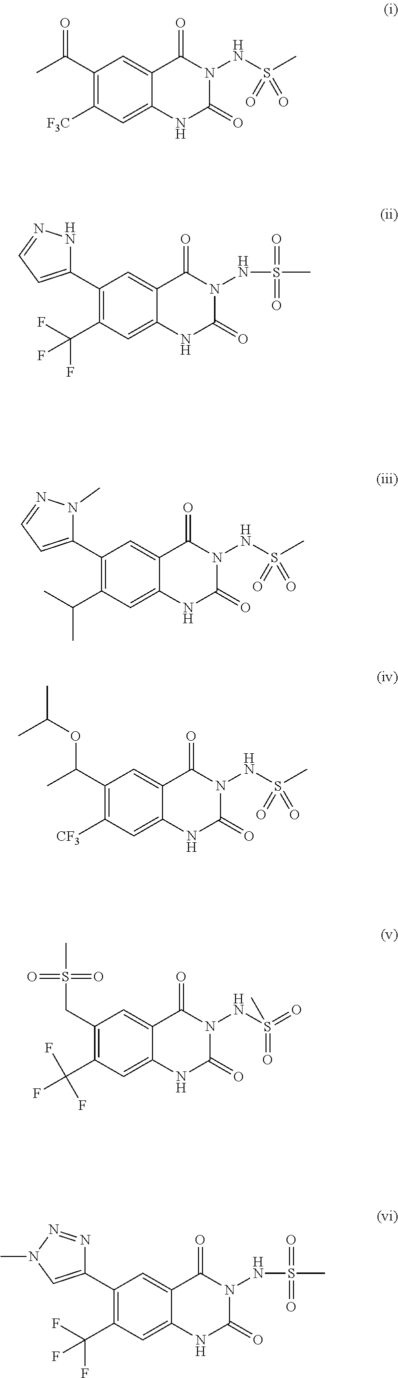

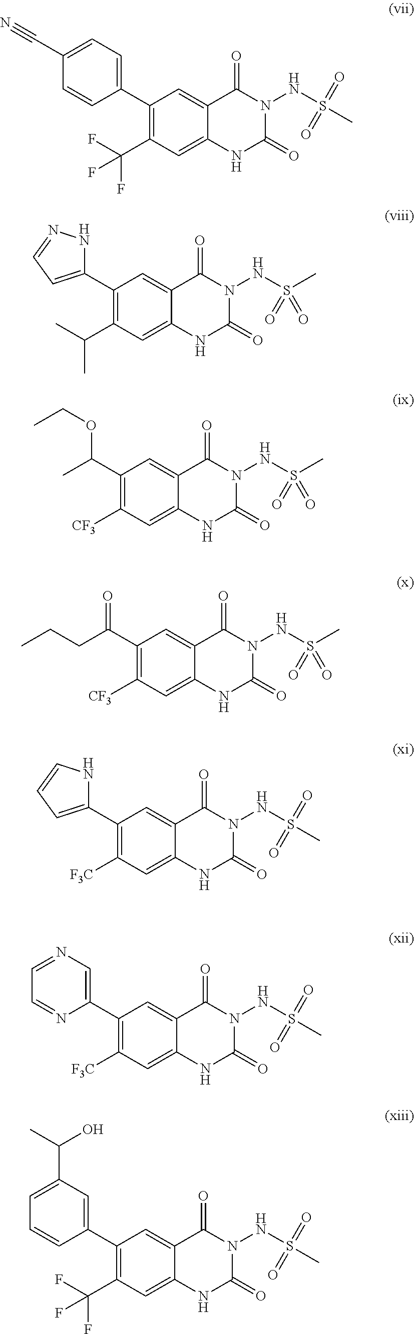

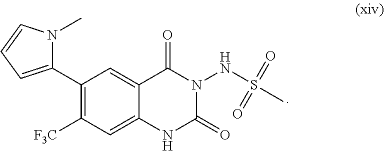

3. A tablet of claims 1, wherein the active ingredient is in an amount of from 2.5% to 30% by weight of the tablet, calculated excluding any coating, and the hydroxypropylcellulose is in an amount of from 2% to 10% by weight of the tablet, calculated excluding any coating.

4-7. (canceled)

8. A tablet of claim 1 which further comprises a water soluble filler, e.g. lactose monohydrate, in an amount of from 20% to 40% by weight of the tablet, calculated excluding any coating.

9. A tablet of claim 1 which further comprises a water insoluble filler, e.g. microcrystalline cellulose, in an amount of from 25% to 50% by weight of the tablet, calculated excluding any coating.

10. A tablet of claim 1 which further comprises a disintegrant, e.g. sodium starch glycolate, in an amount of from 2.5% to 15% by weight of the tablet, calculated excluding any coating.

11-13. (canceled)

14. A tablet of claim 1 wherein the hydroxypropylcellulose has a nominal molecular weight of from 70-100 kDa, e.g. 80 kDa.

15. (canceled)

16. A process for making a tablet comprising an active ingredient selected from compounds of Formula (I) and salts thereof, characterised in that the tablet is made using wet granulation and that the internal phase ingredients comprise a hydroxypropylcellulose as well as the active ingredient.

17. A process of claim 16, wherein the wet granulation uses water in an amount of from 25% to 40% by weight of the solid constituents of the internal phase, and optionally in an amount of from 30% to 35% by weight of the solid constituents of the internal phase.

18. A process of claim 16, wherein the internal phase comprises the active ingredient in an amount of from 2.5% to 30% by weight of the tablet, calculated excluding any coating, and the hydroxypropylcellulose in an amount of from 2% to 10% by weight of the tablet, calculated excluding any coating.

19-21. (canceled)

22. A process of claims 16 wherein the internal phase further comprises a water soluble filler, e.g. lactose monohydrate, in an amount of from 20% to 40% by weight of the tablet, calculated excluding any coating.

23. A process of claim 16 wherein the internal phase further comprises a water insoluble filler, e.g. microcrystalline cellulose, in an amount of from 15% to 40% by weight of the tablet, calculated excluding any coating.

24. A process of claim 16 wherein the internal phase further comprises a disintegrant, e.g. sodium starch glycolate, in an amount of from 1.25% to 7.5% by weight of the tablet, calculated excluding any coating.

25-28. (canceled)

29. A process of claim 16 which comprises:
preparing a wet granulate of an internal phase by combining water and the solid constituents of the internal phase, said solid constituents comprising the hydroxypropylcellulose and the active ingredient, and granulating the combination of the water and the solid constituents;
drying the granulate;
blending the dry granulate with the constituents of an external phase to form a tabletting mixture; and
compressing the tabletting mixture.

30. A process of claim 29, which further comprises film-coating the tablet.
